(19) United States
(12) Patent Application Publication
(10) Pub. No.: US 2015/0140565 A1
(43) Pub. Date: May 21, 2015

(54) Method for Digesting Biological Cells

(71) Applicant: Robert Bosch GmbH, Stuttgart (DE)

(72) Inventors: Uwe Iben, Gerlingen (DE); Peter Rothacher, Bruchsal (DE); Robert Giezendanner-Thoben, Gerlingen (DE)

(21) Appl. No.: 14/394,091

(22) PCT Filed: Mar. 7, 2013
(86) PCT No.: PCT/EP2013/054628

§ 371 Date: Oct. 12, 2014

(30) Foreign Application Priority Data
Apr. 13, 2012 (DE) 10 2012 206 064.2

(51) Int. Cl. C12N 1/06; C12M 1/00; C12N 13/00

(52) U.S. Cl. CPC C12N 1/06; C12N 13/00; C12M 47/06
USPC 435/6.12; 435/173.7; 435/6.1; 435/306.1; 435/4

(57) ABSTRACT

A method for digesting biological cells in a reaction container includes treating the cells with pressure pulses. The pressure pulses are generated by at least one eddy current actuator in conjunction with an electric conductor arranged on or in the reaction container.

The present invention relates to a method for disrupting biological cells and to a device for carrying out the method.

### PRIOR ART

Analysis of biological cellular material is carried out in many areas of biology and biochemistry and especially also in medical diagnostics and research; for example, proteins and other cell constituents are studied and characterized. Particular significance is assigned here to studying the genetic material of cells. For example, particular pathogens are detected in many cases by means of analysis of the genetic material, i.e., DNA in particular. In conventional diagnostics, pathogenic organisms are often determined by means of a pathogen culture. The disadvantage here is that such a culture generally requires multiple days and is additionally burdened with a comparatively high probability of error. Other detection methods therefore utilize molecular biology methods. For example, it is possible to replicate and detect pathogen-specific DNA using the so-called polymerase chain reaction (PCR) developed in the eighties.

The polymerase chain reaction is a highly sensitive in vitro method for selectively replicating defined DNA or RNA segments. Exponential amplification, i.e., replication, of the DNA (or RNA) is carried out in, for example, 20 to 40 cycles. Here, the double-stranded DNA is firstly denatured by heat. Subsequently, specific oligonucleotides (primers) attach to complementary regions of the DNA at a low temperature. Lastly, the new DNA is synthesized as a so-called extension or elongation at a slightly higher temperature, with polymerization of deoxyribonucleoside triphosphates taking place along the DNA template with the aid of the temperature-stable enzyme Taq DNA polymerase. By this means, it is possible to detect highly specific gene regions in order to be able to diagnose, for example, a particular microbial family, genus or species.

The genetic material is usually purified before a polymerase chain reaction is carried out. In some cases, this method can even be carried out without prior purification of the cellular material or the genetic material, if the cells to be studied have been destroyed beforehand by treatment at elevated temperatures and the DNA has been released. This is generally only possible with Gram-negative bacteria. The cell membranes of, for example, Gram-positive bacteria or fungi generally cannot be destroyed to a sufficient extent by high-temperature treatment.

Various other methods are therefore known for disrupting cells or for destroying cell membranes. For example, enzymatic lysis of cells, in which the cell membranes are enzymatically degraded, for example through the use of the enzymes proteinase K or lysozyme, is very widespread. Heat-deactivation of the enzyme used and removal of the proteins are subsequently generally required in view of a polymerase chain reaction, since the activity of the Taq polymerase used for the polymerase chain reaction would otherwise be limited. Such purification on, for example, a silica column requires multiple wash and elution steps, and so enzymatic cell disruption is comparatively complex. In the case of other methods, the cell disruption is carried out using ultrasound. This physical method makes it possible to carry out a PCR reaction directly without further purification of the disrupted cells. However, the expenditure in terms of apparatus is relatively high. When polymer-based devices are used, uncontrolled heating of the substrate is additionally frequently unavoidable, and so experimental results may be distorted. Furthermore, the use of lasers for disruption of bacteria has been described. In the case of the so-called laser cavitation method, a laser pulse results in water in the sample being greatly heated to such an extent that a cavitation bubble arises, which ultimately leads to destruction of the organisms in the sample. For this purpose, high-energy pulses in the infrared range are required. Another method is what is known as electroporation. Bacteria are porosified and the contents thereof are made available by means of high-voltage pulses. Especially in the case of salt-containing solutions, this can result in gases which, for example, can lead to small explosions and are difficult to handle.

In order to be able to carry out analyses at high sample throughputs, use is made of so-called biochips, by means of which various analyses, especially also PCR reactions, can be carried out using very small sample amounts and in an automated manner including sample preparation. They are also referred to as a Lab-on-a-Chip system (LOC). Especially for such LOC systems, it is advantageous when the processing of the samples and the subsequent analysis can be carried out in few steps, and so the method is amenable to automation. Especially in view of Gram-positive bacteria and, for example, fungi, the described methods for disrupting cells are comparatively complex and hardly suitable for an LOC system.

By contrast, it is an object of the present invention to provide a method for disrupting biological cells which can be carried out with little effort and which is readily transferable to an LOC system. In this case, the method is to be suitable in general for bacteria, fungi, viruses and, for example, even eukaryotic cells, the aim being to disrupt the cell membranes reliably without the use of enzymes, providing the cellular material, for example the DNA, for subsequent analytical methods with or without further purification. This object is achieved by a method and a device for carrying out the method as revealed by the independent claims. Preferred embodiments of the method according to the invention or the device according to the invention are revealed by the dependent claims.

### DISCLOSURE OF THE INVENTION

### Advantages of the Invention

The heart of the invention is the treatment of biological cells with pressure pulses. The result of this is that the cell envelopes and, more particularly, the cell membranes are destroyed and the cell components, for example the DNA, can be released from the cell envelope. The pressure pulses preferably have a low wavelength and a high amplitude. The pressure pulses are generated using an impact actuator based on the eddy current principle (eddy current actuator). The eddy current actuator is formed in particular by a coil which acts on an electrical conductor. The electrical conductor is arranged on or in the reaction vessel containing the cells to be disrupted. The cell membranes are thus destroyed via purely physical mechanisms. There is no need for enzymes or other additives, which could interfere with further processing of the sample, for example a PCR reaction. In addition, the use according to the invention of pressure pulses does not lead to excessive heating of the sample or other uncontrollable reactions, which could hamper further analysis. The method according to the invention is therefore suited, especially advantageously, to automation, for example in the form of an LOC system. It is possible according to the invention to disrupt not only Gram-negative bacteria but also Gram-positive bacteria, fungi or spores. In contrast to other physical methods, for example ultrasound and laser, the expenditure in terms of apparatus is distinctly lower for the method according to the invention, making it possible to carry out the method according to the invention in a substantially more cost-effective manner. Because enzymatic and/or chemical additives are not used, further processing and/or analysis is possible with substantially less effort. Moreover, the costs of reagents to be used are also reduced compared to conventional methods.

The method according to the invention is suited, especially advantageously, to so-called μTAS systems (Total Analysis Systems), since in many cases the cells disrupted according to the invention can be used without further purification for, for example, a PCR reaction or other methods. Even in cases where the particular application makes further purification necessary, this is possible with little effort owing to the purely physical disruption of the cells, since, for example, the inactivation of enzymes or the like is not necessary. The method according to the invention is therefore particularly preferably amenable to automation.

The pressure pulses are triggered by a pulsed current in the coil. The application of a current pulse to the coil generates an electromagnetic field. Said electromagnetic field induces in the electrical conductor an eddy current, the electromagnetic field of which is directed toward the primary field. This results in an impulse-type repulsion of the electrical conductor. The changes in position of the electrical conductor are transmitted, especially via the wall of the sample vessel or reaction container, to the liquid in the container. The pressure waves induced by the movement of the wall spread out in the liquid and are reflected at the container boundaries. The pressure waves cause cavitation bubbles and tensions which lead to rupture of the cell envelopes, and so the cells are disrupted.

The treatment with pressure pulses and further processing of the sample, for example a subsequent purification and/or analytical method, can be carried out in the same reaction vessel. This is possible because the disruption method according to the invention is purely physical and relevant analytical reactions, for example PCR reactions, are possible in many cases without further sample preparation. Even if further purification or, for example, enrichment of the genetic material is necessary, this can be carried out in the same reaction vessel with little effort, for example by using a suitable filtration matrix. Depending on the particular application, it may also be preferable to carry out the cell disruption and the further sample processing in separate vessels or containers.

Especially advantageously, the cell disruption according to the invention and further sample processing possibly carried out afterwards, for example a purification and/or analytical method, are carried out in a Lab-on-a-Chip system (LOC). As already mentioned, the disruption method according to the invention is especially suited to LOC systems, since the expenditure in terms of apparatus is comparatively low, and sample preparation which may possibly be required after the cell disruption according to the invention is less complex and especially suited to automation. The use of an LOC for carrying out the cell disruption method according to the invention and for subsequently processing and analyzing the samples can be employed with great advantage for, for example, automated diagnostic methods, for example for detecting pathogens.

Preferably, the biological cells for preparing the cell disruption are segregated and/or purified beforehand from a liquid, for example a body fluid. This purification can, for example, be carried out using a filter on which the cells are segregated. Such a filter can be part of the reaction container in which the cell disruption according to the invention subsequently takes place. Alternatively, enrichment and/or purification of the cells can also be carried out beforehand in a separate container.

Because it is especially suited to automation, the method according to the invention is especially advantageously useful in microbial diagnostics. The automation of the method allows, especially in conjunction with LOC systems, a very rapid and cost-effective procedure with little effort and expenditure with regard to personnel. The method according to the invention can, for example, be used in medical laboratories, in food testing, or in research in general.

The invention further encompasses a device for carrying out the described method, in which biological cells are disrupted by means of treatment with pressure pulses. The device comprises at least one reaction container having an electrical conductor arranged thereon or therein. Furthermore, at least one eddy current actuator, more particularly a coil, is assigned to the reaction container. A coil suitable according to the invention comprises, for example, up to 100 windings. The electrical conductor to which the coil is assigned is, in terms of size, preferably matched to the diameter of the coil. The electrical conductor can, for example, be in the form of an annular disk, a plate or a foil. In one embodiment, the electrical conductor is arranged, for example adhesively bonded, welded, laminated or molded, externally on the reaction container. In another embodiment, the electrical conductor can be arranged inside the reaction container by, for example, being inserted in the form of a plate or disk into the vessel. The wall of the reaction container is preferably distinctly more flexible than the electrical conductor, i.e., the plate or disk for example, and so movement or a change in position of the plate or disk leads to deformation of the wall of the reaction container. The wall of the reaction container can, for example, be a comparatively thin plastic film having a thickness of, for example, from 0.5 to 1 mm, and so it acts like a membrane. When a current pulse flows through the coil, for example as a result of the discharge of a capacitor, an electromagnetic field is generated which generates an opposed field in the electrical conductor as a result of the induced eddy current. This leads to repulsion and thus a change in position of the electrical conductor. These pulse-type changes in position are transmitted to the container wall. The comparatively flexible container wall is thereby deformed in a pulse-type manner. These pulses are transmitted to the liquid within the container. The formation of pressure pulses or pressure waves occurs in the liquid. The pressure waves spread out in the liquid at the speed of sound and are reflected at the inner surfaces of the container. The pressure waves cause cavitation bubbles and tensions in the liquid, which ultimately lead to rupture and destruction of the biological envelope membranes of the organisms or cells present in the liquid.

The advantage of arranging the electrical conductor outside the reaction vessel is that this embodiment can be realized with little effort. Moreover, said embodiment reduces the risk of contamination of the cell sample. The advantage of arranging the electrical conductor within the reaction vessel is that attachment or guidance of the electrical conductor is generally not required. If the geometry of the electrical conductor is tailored to the shape of the reaction containers, the electrical conductor can be positioned by insertion into the container. A particular advantage of this embodiment is that the electrical conductor can be provided with microstructures which trigger shear forces or shear stresses during the as a result of the electromagnetically induced changes in position of the electrical conductor within the container. Said forces can support the destruction of the envelope layers of the biological cells. Said forces can be utilized especially advantageously when the microstructures of the electrical conductor encroach on equal and opposite structures of the inner wall of the container.

In a further embodiment, the electrical conductor can be realized as a plunger or piston. In this case, the entire plunger or piston can be made from electrically conductive material. In other embodiments, the actual electrical conductor is integrated into the plunger or piston. For example, the plunger or piston is itself made of plastic, with a front side of the plunger or piston being provided with an electrically conductive plate or disk. The side containing the electrically conductive plate or disk is facing the eddy current actuator. The other side of the plunger or piston touches the reaction container. One advantage of the plunger- or piston-shaped electrical conductor is that the dimensions of the electrically conductive part of the plunger or piston can be greater than the reaction container to which the electrical conductor is assigned. The eddy current actuator, more particularly the coil, can accordingly be likewise designed to be larger, making it possible to induce stronger forces. A further advantage becomes apparent when comparing the use of an annular disk (without plunger or piston). When using a ring disk, the forces acting in the region of the central opening are only little. When using a plunger or a piston, the change in position caused by the electromagnetically induced change in position of the integrated disk or plate is transmitted to the entire touching surface of the plunger or the piston, improving force transmission.

Preferably, the device for carrying out the method is an LOC system comprising at least one reaction compartment as reaction container. The electrical conductor is arranged in or on said reaction compartment, and so application of current pulses to the eddy current actuator, more particularly the coil, causes, as a result of the induced electromagnetic field, pulse-type changes in position of the electrical conductor which trigger pressure waves and thus tensions in the reaction compartment. This leads to destruction of the biological cells present in the reaction container.

Further processing of the sample and, more particularly, actual analysis of the cell components can in principle take place in the same compartment as the cell disruption according to the invention. In other embodiments, a separate compartment can be provided for this purpose. When carrying out PCR reactions, the corresponding reaction compartment should advantageously be temperature-adjustable. However, the method according to the invention is by no means limited to a combination with PCR methods. For instance, the method according to the invention can be combined with a multiplicity of different methods for detecting different cell components, for example also in combination with immunological methods.

Furthermore, the device can have at least one further compartment intended for detecting reaction products arising, for example, in a PCR reaction carried out after the cell disruption and/or intended generally for detecting cell components. In particular, a conventional array of biological probe molecules suitable for detecting particular molecules from the cell or for detecting, for example, PCR reaction products can be provided for this purpose.

The device according to the invention can be provided with a mechanism for preparing the sample material, for example body fluids, before the disruption of the cells. In particular, a filter can be provided on which cells are segregated from the sample material and enriched before the cell disruption takes place. For example, a fiber pad composed of glass fibers can be integrated into the LOC for this purpose. The sample can be pumped across said fiber pad, and so the cells are segregated on the fiber pad. Subsequent cell disruption can be carried out directly on the fiber pad with said cells, it being possible for the fiber pad or the filter to be part of the reaction container in which the cell disruption and, possibly, also further processing of the cell sample, for example subsequent purification and/or analytical methods, are carried out.

Further advantages and features of the invention are revealed by the following description of exemplary embodiments in conjunction with the drawings. Here, the individual features can each be realized separately or in combination with one another.

### BRIEF DESCRIPTION OF THE DRAWINGS

FIG. 1 diagram of an LOC system for carrying out the method according to the invention in a first embodiment;

FIG. 2 diagram of an LOC system for carrying out the method according to the invention in a second embodiment and

FIG. 3 two embodiments of a reaction vessel according to the invention for disrupting biological cells.

### DESCRIPTION OF EXEMPLARY EMBODIMENTS

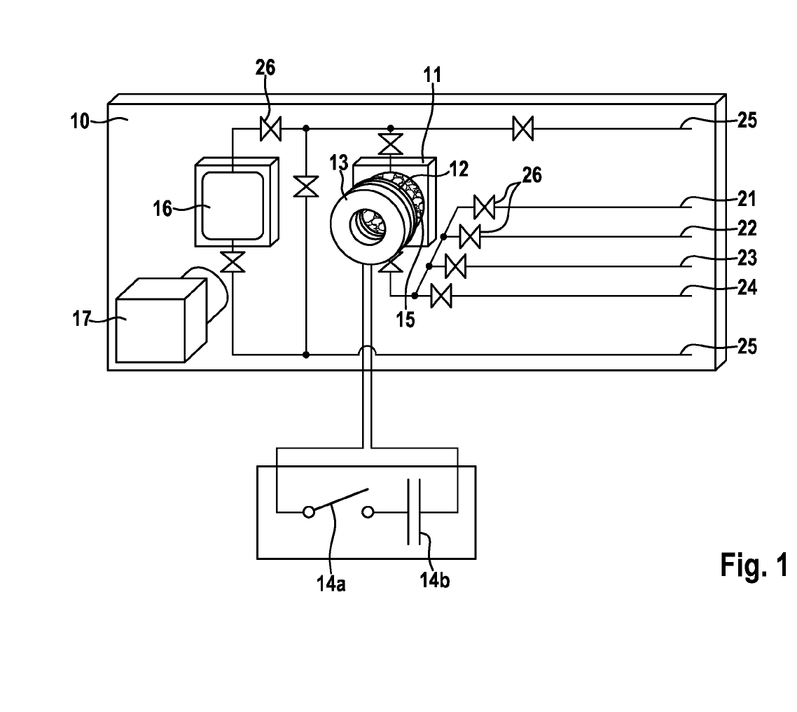

FIG. 1 shows a diagram of an LOC system suited to carrying out the cell disruption according to the invention and to further analysis of the sample. This device is, for example, an injection-molded, polymer LOC inserted into a corresponding operated instrument controlled by, for example, a microprocessor. The LOC can be provided as a disposable article, since, especially for medical applications, this avoids problems related to sterility and to contamination. The LOC 10 comprises a reaction container or reaction compartment 11. The sample containing the cells to be studied is introduced into the container 11. An electrical conductor 12 which, in this embodiment, is in the form of a punched disk composed of electrically conductive material is arranged in the container 11. The electrical conductor should have very good electrical conductivity. A suitable material is, for example, copper, but other metals or alloys can also be used. A coil 13 matching, in terms of its dimensions, the geometry of the electrical conductor is assigned to the reaction compartment 11 as an eddy current actuator. The coil 13 is connected via a switchable electrical contact 14a to a capacitor 14b. When a current pulse is applied to the coil 13, e.g., by discharging a capacitor, this induces in the electrical conductor 12 an eddy current, the electrical field of which is directed toward the primary field. This leads to repulsion of the electrical conductor. The change in position of the electrical conductor caused by the repulsion triggers deformation of the wall of the reaction container 11, which in turn leads to the formation of a pressure wave which spreads out in the sample liquid at the speed of sound. The pressure wave is reflected at the inner boundaries of the reaction compartment 11. Depending on the pressure amplitude, tensions are thereby generated in the liquid, which lead especially to local cavitations. The cavitation bubbles can subsequently collapse and generate further pressure surges. The tensions, pressure waves and pressure surges in the liquid of the reaction compartment that are caused by what has just been described, possibly in combination with shear stresses, lead to rupture of the cell envelopes, i.e., of the cell membranes in particular. The pressure pulses thus generated (eddy current principle) are notable in particular for a low wavelength and a high amplitude. As a result of said pressure pulses, the cell envelopes are destroyed and the material present in the cells, i.e., including the genetic material, is released and can be used for further studies.

The electrical conductor in the form of a plate, disk or foil can be arranged externally on the wall of the reaction container. In other embodiments, the plate, disk or foil can be provided inside the reaction container 11. The reaction compartment or the reaction container can, for example, be provided with an electrically conductive foil composed of copper, aluminum or other metals, for example by adhesive bonding, lamination or welding. The eddy current actuator, i.e., more particularly the coil 13, is preferably mounted in immediate proximity to the electrical conductor, with the distance preferably being distinctly shorter than 1 mm in order to achieve high efficiency with the electromagnetically induced impacts. When current is applied to the eddy current actuator, the coil generates an electromagnetic field, which in turn leads to repulsion of the electrical conductor. These pulse-type changes in position of the electrical conductor are transmitted to the wall of the reaction container, with the elasticity of the thin wall leading to deformations in the wall, which are transmitted as pressure waves to the liquid in the container. The wall of the reaction container can, for example, consist of a polymer material having a wall thickness of approximately 0.5 to 1 mm.

In order to segregate the biological cells from the sample liquid, a filter 15 can be integrated into the LOC 10 and, more particularly, into the reaction compartment 11. Using the filter 15, it is possible to enrich the cells from the sample liquid and to provide them for the cell disruption according to the invention. Furthermore, the filter 15 advanageously allows, for example, washing of the cells.

The LOC 10 comprises various lines or channels intended for the flow of the various liquids required for carrying out the cell disruption method and for subsequent purification and/or analytical methods. The sample can in particular be introduced via the line 21. For instance, a liquid to be studied, for example a body fluid containing bacteria or pathogens to be analyzed, can be pumped into the reaction compartment via the line 21. Possible body fluids are, for example, blood, urine, sputum, serum, plasma, lymph, suspended smears, bronchoalveolar lavage samples, etc. The liquid sample can firstly be pumped across the filter 15, which, for example, consists of glass fibers. Suitable glass fibers of the pad can, for example, have a thickness of from 0.5 to 10 μm. Here, the bacteria or pathogens are segregated on the fiber pad and can be enriched in this process. Subsequently, it is, for example, possible to carry out washing with buffer (e.g., 2 ml), which is supplied via the line 22. The liquids which have passed through can in particular be discharged as waste via the line 25. The fluid management of the various liquids is achieved with the aid of various valves 26, which are provided in the various lines 21 to 25.

Before the actual disruption of the cells, the reaction compartment 11 can, for example, be prefilled with a PCR master mix via the line 23. Such a PCR master mix contains in particular a mixture of nucleotides, primers, Taq polymerase and buffer. Furthermore, if a filter as described here is used, a substance for blocking the filter surface can be present, for example BSA, PEG, PPG or the like. After cell disruption has taken place in the above-described manner, the PCR reaction can be carried out using these substances, it being additionally possible to add further substances and/or buffers via the line 24. Since the various steps of the PCR reaction must be carried out at particular temperatures, the boundary of the reaction compartment 11 is designed to be temperature-adjustable, making it possible to control the temperature within the reaction compartment accordingly and to subject the reaction compartment to the thermal cycles customary for the PCR reaction. Heating elements which can be used and which can be placed externally on the reaction compartment are, for example, Peltier elements, microhotplates or convective heating and cooling elements, possibly also in combination.

During the temperature cycles, the DNA, for example the pathogen-specific DNA, is replicated through the use of suitable primers, as already eludicated at the start. If a filter element 15 is used, the DNA can be subsequently eluted from the filter element 15. For example, it is possible after 20 cycles to isolate 10¹¹ DNA molecules in a 50 μl sample volume from 10 ml of urine containing 10⁵ pathogens. In a similar fashion, the cell disruption method according to the invention can, for example, also be used for amplification of specific RNA segments.

After amplification has taken place, the amplified DNA or the solution containing said amplified DNA can be transferred to a further compartment 16 on the LOC. The reaction products can be detected here. Auxiliary substances which may possibly be required can be supplied. The detection can, for example, be achieved by means of a customary DNA array. Furthermore, it is, for example, possible to provide electrophoretic separation of the reaction products with subsequent visualization. A camera with illumination 17 can be used. It is also especially advantageous, for example, to use what is known as real-time PCR, in which detection and analysis of the reaction products take place as early as during the amplification.

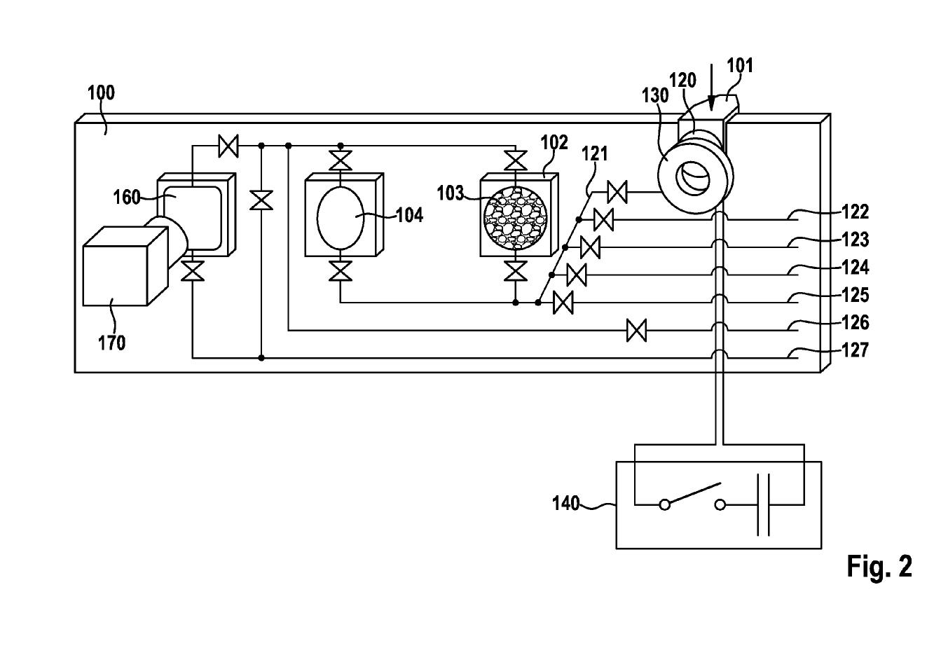

FIG. 2 shows a further embodiment of an LOC system 100 suited to carrying out the cell disruption method according to the invention. In said embodiment, the cell disruption and the further processing of the sample take place in separate compartments. The sample, i.e., for example a body fluid containing the pathogen to be detected, is introduced into a sample reservoir 101. The reservoir is provided with an electrical conductor, more particularly with a metal layer 120. An electrical coil 130 connected to a switchable current source 140 is assigned to the sample reservoir 101. The pressure pulses for disrupting the cells are generated with the aid of the electrical coil 130 as eddy current actuator in conjunction with the electrical conductor, i.e., more particularly the metal layer or metal disk 120. The electrical conductor is arranged, for example adhesively bonded, on the external side of the sample reservoir (reaction container). The current pulse in the electrical coil 130 generates an electromagnetic field. This induces in the electrical conductor an eddy current, the electrical field of which is opposed to the primary field, leading to repulsion of the electrical conductor 120. These pulse-type changes in position of the electrical conductor 120 are transmitted to the elastic vessel wall and generate pulse-type changes in pressure (induction impacts) in the reaction container 101. The tensions, pressure waves and pressure surges in the liquid in the sample reservoir 101 that are ultimately caused by what has just been described lead to destruction of the cell membranes and thus to disruption of the cells. The sample reservoir 101 in the form of a container in which the cell disruption takes place can, as shown here, be part of the LOC system. In other embodiments, another possibility is that said container is provided separately and is, for example, connected via a line to the LOC for further purification and/or processing of the cell sample.

The disrupted sample material can be transferred from the sample reservoir 101, via a line 121, to a further compartment 102 in which purification of the sample material can be carried out. For example, it is possible to integrate in the compartment 102 a silica matrix 103 by means of which the genetic material, more particularly the DNA, from the sample is purified of impurities, cell fragments, etc. The purified genetic material is subsequently transferred to a further compartment 104. A further reaction, for example a PCR reaction, can take place here. The corresponding reagents for the purification in the compartment 102 and for the PCR reaction in the compartment 104 are supplied via the lines 122 (wash buffer), 123 (elution buffer), 124 (PCR master mix) and 125 (hybridization buffer). After the PCR reaction has taken place, the PCR products can be transferred to a further compartment 160 in which the reaction products are detected. In this case, a camera with illumination 170 can be used, comparable to the embodiment in FIG. 1. For a wash buffer which may possibly be required, a line 126 and, for the waste, a line 127 can be provided. The compartment 104 intended in particular for carrying out a PCR reaction is temperature-adjustable in order to be able to carry out the various thermal cycles for the reaction.

The cell disruption according to the invention is combinable with a multiplicity of analytical methods. The PCR method elucidated in detail here is merely one of various possible analytical methods.

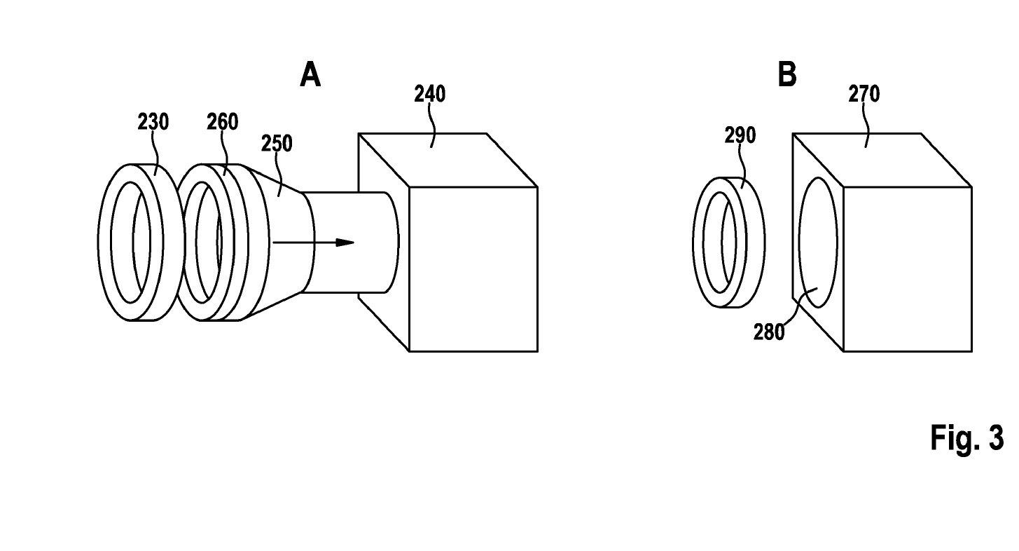

FIG. 3 shows further details of possible embodiments of a device for carrying out the cell disruption according to the invention, with a juxtaposition of an electrical conductor as plunger (subfigure A) and an electrical conductor as disk (subfigure B) being shown. Subfigure A shows the electrical conductor in the form of a plunger 250 which, on the side facing away from the sample reservoir 240, is provided with a ring disk-shaped metal plate 260 as the actual electrical conductor. The metal plate 260 is, for example, adhesively bonded to the plunger 250. The plunger 250 itself is, for example, formed as a solid injection-molded plastic part. Facing the metal plate 260 is a coil 230. The coil 230 and the ring disk 260 match each other in terms of their size. The reservoir 240 contains a suspension of cells which are to be disrupted according to the invention.

Application of a current pulse to the coil 230 establishes an electromagnetc field, resulting in the metal plate 260 together with the plunger 250 being repelled in the direction of the arrow. As a result, electromagnetically induced impacts are triggered onto the elastic wall of the sample reservoir 240, leading to pulse-type pressure wave formation within the liquid of the sample reservoir 240. These pressure waves ultimately lead to tensions in the sample liquid in the reservoir 240, which lead to destruction of the envelopes of the cells in the cell suspension. Subfigure B shows a comparable reservoir 270 containing a cell suspension. The reservoir 270 is provided with an externally arranged metal disk 280 as electrical conductor. In conjunction with application of current to the coil 290, the metal disk 280 causes pressure waves within the liquid present in the reservoir 270, generating the pressure pulses required for destruction of the cell envelopes. The coil 290 is somewhat smaller compared to the coil 230 from subfigure A, since the coil, in terms of its size, matches the size of the metal disk 280 and has approximately the same diameter or a somewhat smaller diameter. Consequently, the acting forces in the embodiment in subfigure B are somewhat weaker compared to subfigure A.

The invention claimed is:

1. A method for disrupting biological cells in a reaction container comprising:
treating the cells with pressure pulses, the pressure pulses being generated by at least one eddy current actuator in conjunction with an electrical conductor arranged on or in the reaction container.

2. The method as claimed in claim 1, wherein a current pulse applied to the eddy current actuator generates an electromagnetic field, resulting in a change in position of the electrical conductor and generating a pressure pulse in the reaction container.

3. The method as claimed in claim 1 wherein the treatment with pressure pulses and further processing of the cell sample are carried out in the same reaction container or in separate reaction containers.

4. The method as claimed in claim 1, wherein one or more of the treatment of the cells with pressure pulses and further processing of the cell sample are carried out in a Lab-on-a-Chip system.

5. The method as claimed in claim 1, wherein cell disruption preparation involves the cells being segregated and/or purified from a liquid, the cells being segregated by a filter.

6. The method as claimed in claim 1, wherein the method is used for a microbial diagnostics procedure.

7. A device for carrying out a method for disrupting biological cells by treatment with pressure pulses, comprising:
at least one reaction container including an electrical conductor; and
at least one eddy current actuator configured as a coil, the at least one eddy current actuator being assigned to the reaction container and configured to generate an electromagnetic field.

8. The device as claimed in claim 7, wherein the electrical conductor is arranged externally on the wall of the reaction container or in the reaction container.

9. The device as claimed in claim 7, wherein the device is a Lab-on-a-Chip system having at least one reaction compartment configured as the reaction container.

10. The device as claimed in claim 7, wherein the reaction container is further configured for the further processing of the cell sample, the reaction container being heatable.

11. The device as claimed in claim 7, further comprising at least one further reaction compartment configured for the further processing of the cell sample, the further reaction compartment being heatable.

12. The device as claimed in claim 8, wherein the Lab-on-a-Chip system comprises at least one further compartment configured to detect one or more of reaction products and cell components.

13. The device as claimed in claim 8,
wherein the Lab-on-a-Chip system comprises a filter configured to segregate cells.
